(19) United States
(12) Patent Application Publication
(10) Pub. No.: US 2012/0258481 A1
(43) Pub. Date: Oct. 11, 2012

(54) CROSS-LINKING ATP ANALOGS

(75) Inventor: Mary Kay Pflum, Northville, MI (US)

(73) Assignee: WAYNE STATE UNIVERSITY, Detroit, MI (US)

(21) Appl. No.: 13/353,346

(22) Filed: Jan. 19, 2012

Related U.S. Application Data
(60) Provisional application No. 61/434,850, filed on Jan. 21, 2011.

(51) Int. Cl. C07H 19/20; C12Q 1/48

(52) U.S. Cl. 435/15; 536/26.26

(57) ABSTRACT

An adenosine 5′-triphosphate analogs modified at the gamma-phosphate with a reactive reagent. A method of forming the analog by activating a 4-amino benzoic acid, incubating the activated acid to obtain an amine, and coupling the amine with ATP in the presence of water soluble EDCI. A method of detecting the efficacy of a therapeutic by adding a gamma-phosphate modified ATP analog to a protein substrate, reacting the target proteins with the ATP analog, and analyzing the resulting cross-linked product, wherein the amount of product present correlates to the efficacy of the therapeutic is also provided.

### CROSS-REFERENCE TO RELATED APPLICATIONS

The present application claims the benefit of priority to U.S. Provisional Patent Application Ser. No. 61/434,850, filed Jan. 20, 2011, the entire contents of each application being hereby incorporated into the present application by reference.

### BACKGROUND OF THE INVENTION

1. Field of the Invention

The present invention relates to certain biologically active ATP analogs. More specifically, the present invention relates to biologically active ATP analogs, their synthesis, their use in pharmaceutical compositions, and methods of use thereof.

2. Description of the Related Art

Proteomics has come of interest over the last few years. While proteomics is more complex than genomics, the study of proteins gives more accurate pictures of cell biology than studying mRNA. The field of proteomics is very broad and involves areas such as, for example, protein profiling by the use of two-dimensional gel electrophoresis and mass spectrometry to study proteins expressed in the cell, protein-protein interaction using yeast two-hybrid method, pathway analysis to understand signal transduction and other complex cell processes, large scale protein folding and 3-D structure studies and high-throughput expression and purification of proteins', cellular expression during metabolism, mitosis, meiosis, in response to an external stimulus, e.g., drug, virus, change in physical or chemical condition, involving excess or deficient nutrients and cofactors, stress, aging, presence of particular strains of an organism and identifying the organism and strain, multiple drug resistance, protein-DNA interactions, protein-peptide interactions, and the like. It is necessary to have a means for identifying a large number of proteins in a single sample, as well as providing some quantitation of the different proteins being detected.

As the human genome is elucidated, there will be numerous opportunities for performing diagnostic procedures relating to the coding sequences of genes. One major function of genes is to generate proteins, which play a major role in the work carried out in a cell. Because the protein functions in a cell are dynamic, the structure, concentration, location, and so forth of a particular protein at a particular point in time is constantly changing. Analysis of protein expression patterns is the subject of ongoing genomics projects. Studies of physiologically active forms of proteins and their spatial and temporal interaction in the cell Is an important aspect of the overall study.

One post-translational modification of proteins is the addition or removal of phosphate groups. Protein phosphorylation and de-phosphorylation reactions have been established as major components of metabolic regulation and signal transduction pathways. Variations in protein phosphorylation provide the predominant means of enzymatic regulation now known in biological systems, especially in the regulation of signal transduction from cell surface receptors. Reversible phosphorylation is important for transmitting regulatory signals, including proliferative ones, in all living cells. To understand the molecular basis of these regulatory mechanisms, it is necessary to identify the specific amino acid residues that become phosphorylated. By identifying the substrates and sites of phosphorylation, diagnostic tools may be developed for some tumors and the modification of the process itself could be a target for therapeutic intervention.

Polypeptides such as growth factors, differentiation factors and hormones are crucial components of the regulatory system that coordinates development of multicellular organisms. Many of these factors mediate their pleiotropic actions by binding to and activating cell surface receptors with an intrinsic protein tyrosine kinase activity. Changes in cell behavior induced by extracellular signaling molecules such as growth factors and cytokines require execution of a complex program of transcriptional events. To activate or repress transcription, transcription factors must be located in the nucleus, bind DNA, and interact with the basal transcription apparatus. Accordingly, extracellular signals that regulate transcription factor activity may affect one or more of these processes. Most commonly, regulation is achieved by reversible phosphorylation. Phosphorylation of a transcription factor by several different kinases (or by a kinase linked to more than one pathway) is a simple mechanism that allows different signals to converge at the same factor.

Since the mid-1950's, the reversible phosphorylation of amino acid side chains, been acknowledged as a key regulatory mechanism in cells. Approximately 30% of known proteins are phosphorylated. Protein phosphorylation is controlled by the equilibrium activity of kinase enzymes that phosphorylate proteins and phosphatase enzymes that de-phosphorylate proteins. Kinases, phosphatases, and their phosphorylated substrate proteins are found ubiquitously in vivo and are involved in a variety of disease states. As a result, development of facile approaches to characterize kinase and phosphatase activities, along with their phosphorylated substrates, is an important aspect of disease characterization and drug design.

The current methods available for identifying phosphorylated proteins or the kinases mediating the phosphorylation are generally difficult and not rigorous. The traditional method of isolating ³²P-labeled phosphoproteins is accessible to any scientist with knowledge of cell biological techniques. Unfortunately it is time-consuming, hazardous, and insufficiently sensitive to detect all fragments. Whereas ³²P-labeling is useful for studying a single known protein, multiple proteins are not easily assessed using this method, as required for proteomics research. Alternatively, mass spectrometric analysis of phosphoprotein samples has been successfully used to analyze complex protein mixtures. Unfortunately, mass spectrometric analysis does not necessarily assess all phosphorylated species; where the presence of a phosphorylated peptide ion is certainly evidence of its existence in the protein, the absence of a peptide ion does not allow conclusions to be drawn about its existence.

To increase the probability for quantitative phosphorylation site identification using mass spectrometry, many strategies have incorporated a phosphoprotein purification step prior to mass analysis. For example, there are several strategies that allow chemical tagging of a phosphorylated amino acid. Unfortunately, the methods suffer from low yields due to multi-step chemical schemes and subsequent trypsin digestion or require chemical synthesis of solid phase resins that are not commercially available.

These limitations have stalled the use of these innovative techniques. Alternatively, the interaction of a phosphate with a metal binding reagent attached to a solid phase has been developed and several commercially available “kits” utilize this technology. In this case, the technology is limited by the relatively nonselective interaction with phosphates with metal binding agents compared to the covalent modifications.

In addition to the difficulties related to characterizing phosphorylated proteins, monitoring kinase and phosphatase activity has also met challenges. In the case of kinases, which are a significant drug target in the pharmaceutical world, monitoring the activity of the roughly 500 kinases in a cell has been an unresolved task. As a result, identifying drugs targeting a specific single kinase has been stalled due to the in ability to screen all kinase proteins. Therefore, new methods that allow facile monitoring of kinases activity would aid in drug design efforts.

### SUMMARY OF THE INVENTION

According to the present invention there is provided a method and composition for use in tagging phosphoproteins for subsequent analysis by mass spectrometry, gel electrophoresis, or other similar method. The phosphorylation of proteins in vivo requires a ternary complex comprised of 1) a protein substrate to be phosphorylated, 2) a kinase protein enzyme to catalyze the phosphate transfer reaction, and 3) an adenosine 5′-triphosphate (ATP) co-substrate to provide the phosphate group to be transferred.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1. a) Chemical structures of ATP 1 and γ-phosphate modified ATP analogs ATP-ArN3 2 and ATP-biotin 3. b) Kinase-mediated phosphorylation of a hydroxyl-containing amino acid (serine is shown) with ATP-ArN3 2 produces a photocrosslinking protein product 5. Cleavage of the phosphoramidate bond is achieved with acid (50% TFA). c) UV irradiation during kinase catalysis covalently crosslinks kinase and substrate through the ATP-ArN3 probe.

FIG. 2. Kinase-catalyzed photocrosslinking with peptide substrates. CK2 (a) or Abl (b) kinases were incubated with rhodamine-labeled CK2 substrate peptide 14 (Rho-14) or ROX-labeled Abl substrate peptide 13 (ROX-13), respectively, in the presence of ATP 1 or ATPArN3 2 before separation by SDS-PAGE and visualization by in gel fluorescence scanning (a and b, top), coomassie staining (a, bottom), or silver staining (b, bottom). Reactions were incubated either with or without exposure to 365 nm light (UV). Trifluoroacetic acid was added to a final concentration of 50% after crosslinking (acid). The gels are representative of at least three independent trials.

FIG. 3. Kinase-catalyzed photocrosslinking with full-length α-casein. CK2 kinase was incubated with full-length α-casein in the presence of ATP 1 or ATP-ArN3 2 before separation by SDS-PAGE and visualization by coomassie staining (a), or a CK2 antibody (b). The expected 68 kDa crosslinked complex is indicated as CK2-α-casein. The contents of each reaction are indicated below each lane (see FIG. 2 legend). The gels are representative of at least three independent experiments.

FIG. 4. Kinase-catalyzed affinity crosslinking with full-length α-casein. CK2 kinase was incubated with full-length α-casein in the presence of ATP 1 or ATP-IAc 3 before separation by SDS-PAGE and visualization by coomassie staining (a), or a CK2 antibody (b). The expected 68 kDa crosslinked complex is indicated as CK2/α-casein crosslink. The contents of each reaction are indicated below each lane, with the concentration of the ATP-IAc indicated in μM. The gel is representative of at least three independent experiments.

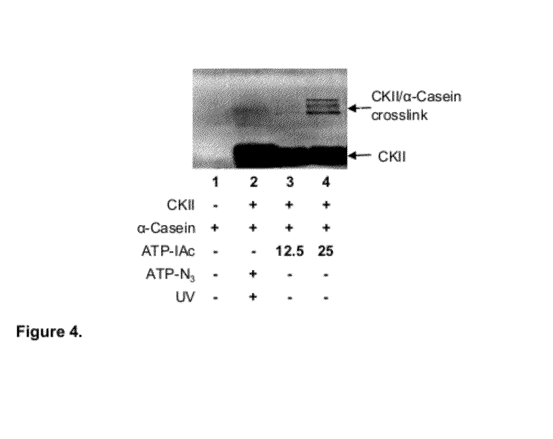

### DESCRIPTION OF THE PREFERRED EMBODIMENTS

The present invention provides a composition of matter, in the form of adenosine 5′-triphosphate (ATP) analogs modified at the gamma-phosphate with crosslinking groups. The composition can be used for tagging phosphorylated proteins using the ATP analogs and kinase enzymes.

The present invention provides reagents and methods for tagging the phosphorylated proteins. Proteins can be post-translationally modified such that they contain phosphate groups at either some, or all serine, threonine, tyrosine, histidine, and/or lysine amino acid residues. In many cases the extent to which a protein is phosphorylated determines it bioactivity, i.e., its ability to effect cell functions such as differentiation, division, and metabolism. Hence, the present disclosure provides powerful methods for diagnosing various diseases and for furthering the understanding of protein-protein interactions.

“Specific” in reference to the binding of two molecules or a molecule and a complex of molecules refers to the specific recognition of one for the other and the formation of a stable complex as compared to substantially less recognition of other molecules and the lack of formation of stable complexes with such other molecules. Preferably, “specific” in reference to binding means that to the extent that a molecule forms complexes with other molecules or complexes, it forms at least fifty percent of the complexes with the molecule or complex for which it has specificity. Generally, the molecules or complexes have areas on their surfaces or in cavities giving rise to specific recognition between the two molecules. Exemplary of specific binding are antibody-antigen interactions, enzyme-substrate interactions, polynucleotide interactions, cellular receptor-ligand interactions, and so forth.

The composition enables the tagging of phosphoproteins for subsequent analysis by mass spectrometry, gel electrophoresis, and other methods known to those of skill in the art. In some instances the use of mass spectrometry analyses might not be desirable. In these instances the spectrophotometric qualities and binding properties of biotin can be exploited. For example, the biotin labeling procedure can be used in conjunction with ultraviolet (UV) spectroscopy to measure the incorporation of biotin which absorbs at 220 nm (Przyjazny A. et al. Anal. Chem. 62:2536-2540, 1990). Fluorescence detection is also can also be used.

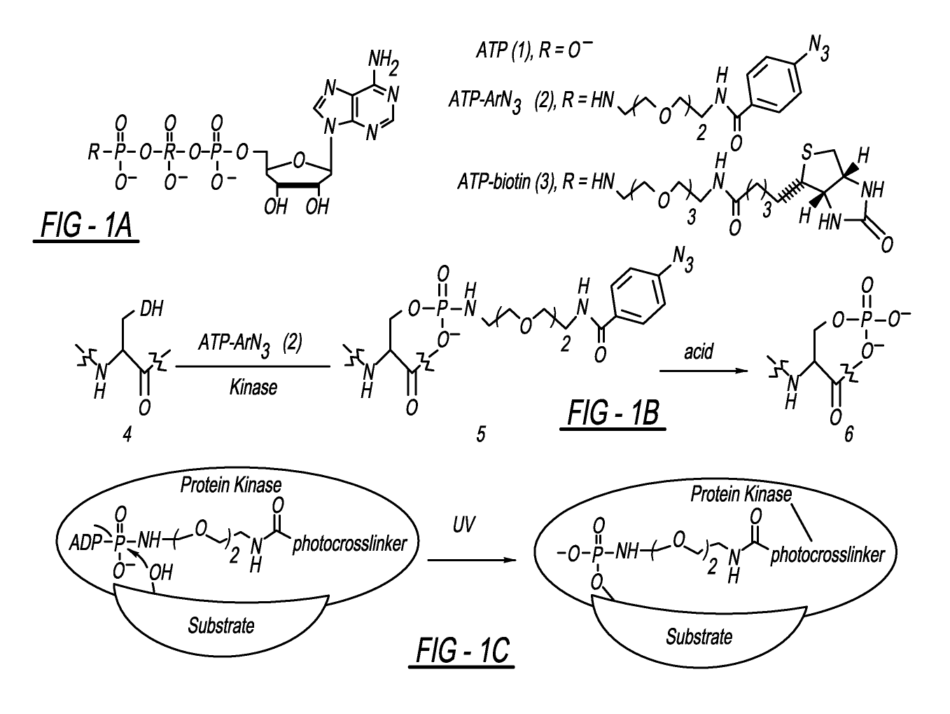

As shown in FIG. 1, phosphorylation of proteins in vivo requires a ternary complex. A ternary complex refers to a protein complex containing three different molecules which are bound together. In structural biology ternary complex can be used to describe a crystal containing a protein with two small molecules bound, for example cofactor and substrate; or a complex formed between two proteins and a single substrate. As used herein, the ternary complex is 1) a protein substrate to be phosphorylated, 2) a kinase protein enzyme to catalyze the phosphate transfer reaction, and 3) an adenosine 5′-triphosphate (ATP) co-substrate to provide the phosphate group to be transferred.

The composition of the present invention can be in the form of a modified ATP analog that includes a reactive reagent attached to the gamma-phosphate. As used herein the term “reactive reagent” is defined to include any group capable of coupling or crosslinking two molecules. Reactive reagents can include any chemical or biology entity that is capable of “reacting”. A reactive reagent can include crosslinking entities, but can also include groups that would catalyze or initiate a reaction but not result in a covalent coupling (an enzyme like horse radish peroxidase (HRP), for example). Below are provided several examples of such ATP analogs. This list is not meant to be exhaustive and is instead provided as examples wherein one of skill in the art could modify and create other ATP analogs using the present invention to create alternative compositions encompassed by the present invention.

First, ATP-arylazide and ATP-benzophenone analogs represent the general compounds where a benzophenone group or an aromatic group containing an azide is attached to the gamma-phosphate of ATP through a linker containing ten or more atoms. These analogs allow the tagging of phosphorylated protein with an aryl azide group. Aryl azides are activated with UV light to form a covalent crosslink with other biomolecules. As a result, aryl azides are a photoreactive crosslinking group. A linker of ten atoms or greater is needed to connect the aryl azide from the ATP to allow the kinase to accept in the enzyme active site the relatively large aryl azide and benzophenone groups on the ATP co-substrate.

Second, ATP-diazirine analogs represent general compounds where a diazirine is attached to the gammaphosphate through any linker of at least one atom. Because diazirines are photo-activated crosslinking reagents, these reagents can crosslink in a manner similar to ATP-aryl azides and ATP-benzophone compounds. However, due to the smaller size of the diazirine group compared to the relatively larger aryl azide or benzophenone, a shorter linker is compatible with the kinase reaction.

Third, ATP-azide or ATP-alkyne analogs represent the general compounds where any azide or alkyne is attached to the gamma-phosphate through any linker of at least one atom. Azide and alkyne groups are useful for subsequent attachment of any desired label (biotin, fluorophores, etc) using known “bioorthogonal chemistry”, such as “click” chemistry or Staudinger reactions. Therefore, these analogs have the flexibility of labeling phosphoprotein with literally any probe of interest.

Fourth, bi-functional crosslinking ATP analogs are ATP analogs with a single crosslinking reagent, bi-functional ATP analogs containing a crosslinking group along with a second chemical entity, such as biotin or its analogs, fluorophores, azides, alkynes, etc can also be utilizes. These bi-functional ATP analogs allow crosslinking in addition to purification/visualization, which enhances the utility of the probes for the applications disclosed herein. The linker connecting the ATP to the functional groups can include any chemical group with any number of atoms.

Fifth, ATP analogs containing an affinity-based crosslinking reagent at the gamma phosphate can also be utilized. Affinity-based crosslinking groups are defined as chemical entities that require binding interactions between the crosslinking molecules to allow crosslinking. Examples of affinity-based crosslinking reagents include, but are not limited to, an alpha-dibromobutadione, alpha-iodoacetamide, alpha-bromoacetamide, or florophosphates. The linker connecting the ATP to the affinity-based crosslinking group can include any chemical group with any number of atoms. The affinity-based crosslinking ATP analogs offer the advantage of requiring binding interactions between molecules as a prerequisite for crosslinking. As a result, these reagents are useful to probe kinase- or phosphorylation-mediated molecular interactions.

To incorporate a chemical tag specifically on a phosphate group and aid in phosphoprotein characterization, analogs of the ATP co-substrate with crosslinking groups attached to the gamma-phosphate of the ATP have been generated (FIG. 1). It was found that 1) the presence of the crosslinking group on the gamma-phosphate of the ATP does not interfere with phosphate group transfer to the protein substrate in the kinase enzyme active site, and 2) the modified phosphate group is transferred with the expected production of a phosphorylated protein substrate and ADP. Therefore, these ATP analogs modified at the gamma-phosphate can be used as reagents for the incorporation of crosslinking groups on phosphorylated amino acids on proteins using kinase activity. Therefore, the invention aids in the characterization of phosphorylated proteins and advance the field of proteomics research. In addition, with kinase enzymes important in disease formation and the target a drug design efforts in the pharmaceutical community, the crosslinking ATP analogs can also allow monitoring of kinase activity.

The use of gamma-phosphate modified ATP analogs provides a novel means of chemically labeling or tagging a protein specifically at a phosphorylated amino acid. Protein phosphorylation is widely accepted as a common means for organisms to influences protein functions in a cell, including a human cell. Therefore, characterizing the phosphorylation of a protein is critical to completely understand biological events associated with disease development. The present method, using crosslinking ATP analogs, allows the selective tagging of a protein at phosphorylated amino acids with a crosslinking group either in the cell or outside of the cell. By labeling or tagging a protein selectively at phosphorylated amino acids with a crosslinking reagent, the phosphorylation protein, associated proteins, and the activities of the enzymes that regulate phosphorylation can be more easily characterized.

FIG. 1 depicts the mechanism of protein phosphorylation by kinases. When ATP is the co-substrate (X=0), a phosphate is transferred to the protein substrate. Gamma-phosphate modified ATP analogs (where X=biotin fluorophores. etc) serve as efficient kinase co-substrates, allowing kinase-catalyzed phosphoprotein labeling. The realization that kinases tolerate ATP analogs as co-substrates is the basis for this invention. The chemical reaction involves nucleophilic attack of the terminal phosphate of ATP (the gamma-phosphate) by the hydroxyl group of the amino acids serine, threonine, or tyrosine resulting in the production of a phosphorylated serine, threonine or tyrosine on the protein substrate.

Additionally, kinases catalyze protein phosphorylation to regulate signal transduction in a cell. As a result, identification of substrates to a particular kinase is a fundamental step toward understanding the signaling cascades in normal and disease states. With over 500 known kinases and a paucity of tools available, identifying genuine kinase substrates has been difficult. New approaches to detect substrates are needed to fully characterize cell-signaling networks. Adenosine 5′-triphosphate (ATP, FIG. 1a) is the universal co-substrate responsible for protein phosphorylation. During phosphorylation, the γ-phosphate of ATP is transferred to serine, threonine and tyrosine residues on eukaryotic protein substrates. The present invention demonstrates that cellular kinases promiscuously accept ATP analogs with γ-phosphate modifications. Specifically, ATP analogs modified at the γ-phosphate with either dansyl or biotin (FIG. 1a) were coupled with cellular kinases to conveniently label peptides and proteins with the dansyl or biotin group for subsequent analysis by mass spectrometry (MS) or gel electrophoresis. Kinase-catalyzed phosphoprotein labeling is a useful enzymatic approach to attach a desired functional probe to a substrate protein.

To address the challenge of identifying substrates, kinase-catalyzed labeling can be combined with photocrosslinking to covalently couple enzyme to substrate. Kinases will accept a photoactivatable ATP analog as a co-substrate (ATP-ArN₃, FIG. 1a) and generate a protein product capable of crosslinking (FIG. 1b). With simultaneous UV irradiation during phosphorylation, the photocrosslinker will covalently link the substrate to kinase, producing a high molecular weight protein complex (FIG. 1c). The kinase and substrate in the crosslinked product can be identified using traditional gel electrophoresis and western blotting. A powerful feature of the photocrosslinking ATP analog is that it requires phosphorylation for crosslinking. As a result, all crosslinked complexes necessarily contain a kinase substrate. The photocrosslinking ATP-ArN₃ analog would be the first phosphorylation-dependent kinase-substrate crosslinking reagent.

To study a photocrosslinking ATP analog, ATPArN₃ (FIG. 1a) was synthesized. An aryl azide (ArN₃) was selected as the photocrosslinker since it is small, stable under standard biological conditions, and used previously to photocrosslink proteins. A hydrophilic linker mimicking that in ATP-biotin (FIG. 1a) was also chosen. The aryl azide was synthesized starting with commercially available 4-amino benzoic acid (Scheme 1). The acid was activated with N-hydroxysuccinimide (NHS) and further incubated with 1,8-diamino-3,6-dioxaoctane to obtain amine. The final compound was obtained by coupling amine with ATP in presence of a water-soluble carbodiimide (EDCI) while controlling the pH at 5.6-5.8. A more detailed explanation is provided in the examples.

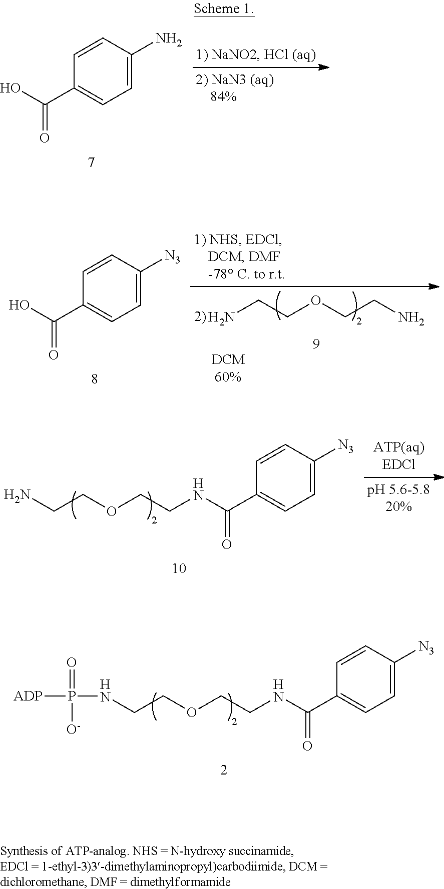

The composition of the present invention has many anticipated commercial uses. For example, once the crosslinking reagent has been attached to the kinase substrate via kinase catalysis, the crosslinking reagent will link the substrate to the kinase via a covalent bond. As a result, the crosslinking ATP analogs provide a novel approach to identify the kinase associated with a specific substrate. Because no other method is available for kinase-catalyzed crosslinking, the cross linking ATP analogs form the foundation of a biochemical kit sold to the biomedical community to easily identify kinase substrates.

For example, the methods and reagents described herein may be used to profile the phosphorylation states of multiple proteins. The proteins may be derived from tissue samples such as tumor samples, body fluids such as urine, saliva, or blood, or cell cultures. Using the present methods to tag proteins, samples containing a heterologous mixture of proteins may be exposed to one or more differentially labeled crosslinking ATP analogs, be substantially isolated, and then subjected to detection based upon the differences in their length and/or weight. Such analysis provides a profile of the phosphorylation states of the proteins from the biological sample.

Additionally, the composition of the present invention can also be used for the identification of phosphorylation-associated biomolecules. After phosphorylated proteins are tagged with a crosslinking group via the ATP analog and a kinase, associated proteins, DNA, or other biomolecules can be identified using established biochemical methods, including SDS-PAGE, chromatography, or mass spectrometric analysis. The kit emerging from this use is similar to that described above, but focuses in on crosslinked proteins that are not kinases.

The composition of the present invention can also be used for the versatile tagging of phosphoproteins. The crosslinking group on the ATP analog can be a bioorthogonal crosslinking reagent (not reactive with natural biomolecules; only reactive with unnatural molecules). In this case, after labeling the phosphoprotein with a bioorthogonal crosslinking group using the ATP-crosslinking analog and kinase, a secondary probe containing a reagent capable of reacting with the bioorthogonal crosslinker can be added to attach a desired label to the protein. The secondary probe can contain the reagent capable of reacting with the bioorthogonal crosslinker along with a desired label, including, but not limited to, biotin, fluorophores, and other labels known to those of skill in the art.

Further, there are disease states that have been correlated with the abnormal phosphorylation of proteins. Abnormal in this context refers to a deviation from normal characteristics. Normal characteristics can be found in a control, a standard for a population, etc. Likewise, abnormal may refer to a condition that is associated with a disease. The term “associated with” includes, for example, an increased risk of developing the disease as well as the disease itself. For instance, a certain abnormality (such as a hyperphosphorylation of tau protein) can be described as being associated with the biological condition of Alzheimer's disease (decrease in bone mass); thus, the abnormality is predictive both of an increased risk of developing Alzheimer's disease and of the presence of Alzheimer's disease.

Abnormal protein modification, such as abnormal protein phosphorylation, refers to the modification of a protein that is in some manner different from expression of the protein in a normal situation. This includes but is not limited to: (1) a mutation in the protein such that one or more of the amino acid residues is different; (2) a short deletion or addition of one or a few amino acid residues to the sequence of the protein; (3) a longer deletion or addition of amino acid residues, such that an entire protein domain or sub-domain is removed or added; (4) expression of an increased amount of the phosphorylated protein, compared to a control or standard amount; (5) expression of an decreased amount of the phosphorylated protein, compared to a control or standard amount; (6) alteration of the subcellular localization or targeting of the protein; (7) alteration of the temporally regulated expression of the protein (such that the protein is expressed when it normally would not be, or alternatively is not expressed when it normally would be); and (8) alteration of the localized (e.g., organ or tissue specific) expression of the protein (such that the protein is not expressed where it would normally be expressed or is expressed where it normally would not be expressed), each compared to a control or standard.

Controls or standards appropriate for comparison to a sample for the determination of abnormality include laboratory values, keeping in mind that such values may vary from laboratory to laboratory. Laboratory standards and values may be set based on a known or determined population value and may be supplied in the format of a graph or table that permits easy comparison of measured, experimentally determined values.

Determining that a protein or a set of proteins are displaying an abnormal phosphorylation state can be accomplished using the present invention methods. Specifically, a sample containing the test protein(s) (i.e., proteins that are from a source that is suspected of displaying abnormal phosphorylation levels) is contacted with a composition of the present invention, and in a separate reaction, contacting a control sample with a differentially labeled composition. The samples can then be analyzed to determine if the test sample contains either hyper- or hypo-phosphorylated proteins compared to the control.

The methods described herein can also be used to identify potential therapeutic agents, such as chemical agents, therapeutic nucleic acid molecules, or peptides, that act to alter the phosphorylation state of proteins suspected of contributing to disease. Once an abnormally phosphorylated protein has been identified as contributing to a disease state the methods described herein can be used to screen for compounds that alter the phosphorylation state of the protein. This can be accomplished by contacting a sample, i.e., a sample from a subject, cell culture, or tissue culture that displays the hyper- or hypo-phosphorylated protein to the test compound (i.e., therapeutic). The proteins from the sample can then be isolated and tagged with the crosslinking ATP analogs and analyzed to see if the test compound was capable of altering the phosphorylation state of the protein.

The amount or concentration of the therapeutic may need to be varied in order to determine whether it effectively alters the phosphorylation level (state) of the protein. One of ordinary skill in the art will appreciate what amounts would be necessary or that simple experiments using varying concentrations of the therapeutic can be designed. For example, using concentrates of the therapeutic ranging from about 1 pg/mL to about 1 .mu.g/mL, or about 1 ng/mL to about 1 g/mL identifies the concentration at which the therapeutic compound alters the phosphorylation state of the protein.

The above discussion provides a factual basis for the methods and uses described herein. The methods used with and the utility of the present invention can be shown by the following non-limiting examples.

### EXAMPLES

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

### Recombinant Protein Purification:

Marshak et al, “Strategies for Protein Purification and Characterization. A laboratory course manual.” CSHL Press, 1996.

### Example 1

To test the compatibility of the synthesized ATP-ArN₃ with kinase-catalyzed labeling, peptide substrates containing serine, threonine and tyrosine were incubated with ATP-ArN₃ along with their respective kinase, PKA, CK2 or Abl (Table 1). The products were analyzed by quantitative Matrix Assisted Laser Desorption Ionization—Time Of Flight (MALDI-TOF) MS to determine the efficiency of the labeling, as described previously. Quantitative MS data (Table 1) indicated that each kinase modified its corresponding peptide substrate with 51 to 86% conversion (Schemes S1-S3). The conversion efficiencies observed with ATP-ArN₃ are similar to those observed with ATP-biotin (56 to 80%). These results confirm the ATP co-substrate promiscuity of kinases and prove that peptide substrates can be labeled with a photocrosslinker using ATP-ArN₃.

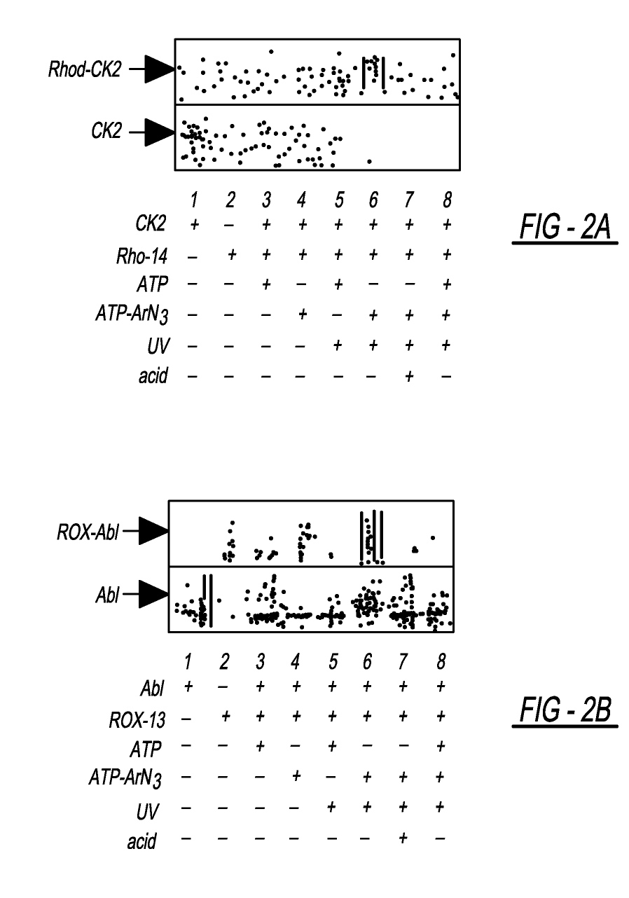

Having validated that ATP-ArN₃ is a co-substrate, use of the photocrosslinker to covalently couple kinase and substrate was assessed. Crosslinking was tested by incubating CK2 kinase and ATP-ArN₃ with rhodamine-labeled CK2 peptide substrate 14 (Rho-RRREEETEEEE, Rho-14). Simultaneously, the reaction mixture was irradiated with 365 nm light to afford a covalent crosslink (FIG. 1c). If kinase-substrate crosslinking occurred, the kinase was expected to become fluorescently labeled, which would be visualized by gel electrophoresis. As expected, in-gel fluorescence scanning showed the presence of fluorophore-labeled CK2 after UV-mediated crosslinking with ATP-ArN₃ and Rho-14 (FIG. 2a, lane 6). As controls, no fluorescence labeling was observed without UV light or with natural ATP (FIG. 2a, lanes 3-5), indicating that the aryl azide and photoactivation are necessary for crosslinking. Addition of competing ATP or unlabeled CK2 substrate peptide into the ATP-ArN₃-mediated crosslinking reaction prevented fluorophore labeling (FIG. 2a, lane 8; Figure S4, lane 5). In addition, no fluorophore labeling was observed with heat denatured CK2 (Figure S4, lane 7), indicating that a kinase active site is required for crosslinking. As a final critical control, the crosslinked product was treated with acid to cleave the phosphoramidate bond bridging the peptide and kinase (FIG. 1c).

As expected, no fluorescence was observed after treatment with acid (FIG. 2a, lane 7; Figure S4, lane 3), establishing that acid incubation is a means of releasing kinase and substrate. These series of experiments affirm that crosslinking is dependent on kinasecatalyzed labeling. In total, the data indicate that kinase-substrate crosslinking is phosphorylation-dependent and requires active kinase, ATP-ArN₃, and UV irradiation.

To validate that phosphorylation-dependent substrate crosslinking is compatible with multiple kinases, the Abl kinase was tested with ROX-labeled Abl peptide substrate 13 (ROXEAIYAAPFAKKK, ROX-13). As seen with CK2, Abl was fluorophore labeled only after UV-mediated crosslinking with ATP-ArN₃ and ROX-13 (FIG. 2b, lanes 3-6). In addition, fluorophore labeling was lost after incubation with acid (FIG. 2b, lane 7), confirming the presence of the phosphoramidate bond. The data indicate that phosphorylation-dependent kinase-substrate crosslinking is compatible with multiple kinases.

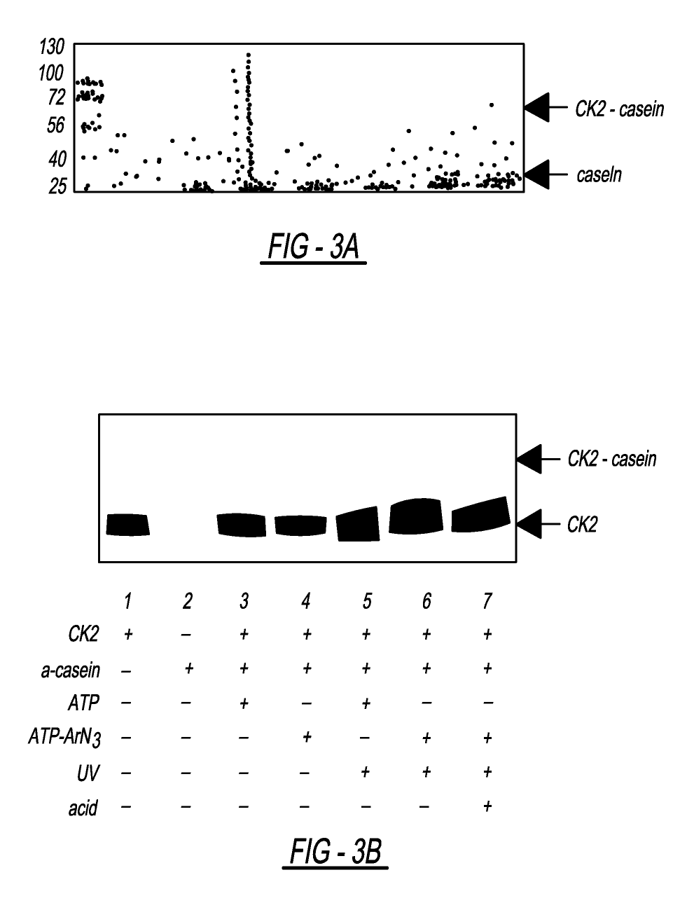

Having established phosphorylation-dependent crosslinking between kinase and peptide substrate, crosslinking with a full-length protein substrate was tested. The α-casein protein has been widely used as a model CK2 substrate in phosphoprotein analysis. Crosslinking was carried out by incubating a mixture of CK2 and α-casein with ATP-ArN₃ in the presence of UV light. The products were separated by SDS-PAGE and analyzed by coomassie staining (FIG. 3a) and western blot analysis (FIG. 3b). Coomassie staining showed an increase in the intensity of a band at roughly 68 kD, which is the expected crosslinked product of the catalytic α-subunit of CK2 (45 kD) and α-casein (23 kD) (FIG. 3a, lane 6). Importantly, immunoblotting showed the presence of CK2 in the 68 kDa complex (FIG. 3b, lane 6). As controls, the crosslinked complex was absent in reactions performed without UV light or with ATP (FIG. 3b, lane 3-5). In addition, the CK2-crosslinked complex was lost after acid treatment (FIG. 3b, lane 7), consistent with cleavage of the phosphoramidate bond and degradation of the α-casein-CK2 complex. These experiments demonstrate that a full-length protein substrate is compatible with phosphorylation-dependent kinase-substrate crosslinking. In conclusion, there was demonstrated the first phosphorylation-dependent kinase-substrate crosslinking strategy. The data indicate that crosslinking requires the ATP-ArN₃ reagent, active kinase, and UV irradiation. Because phosphate transfer is required, a powerful feature of ATP-ArN₃-mediated crosslinking is that a kinase substrate is necessarily present in the crosslinked product. Coupled with mass spectrometric analysis, phosphorylation-dependent kinase-substrate crosslinking can determine the sites of phosphorylation in addition to the effector kinase. Importantly, the crosslinking approach is appropriate for studies with cell lysates or homogenized tissue samples since natural kinase and substrate are utilized. In total, phosphorylation-dependent kinase-substrate crosslinking is expected to aid in the characterization of cell signaling networks.

Photocrosslinking was initiated by irradiating with a handheld UV lamp at 365 nm for 2 hours at 30° C. The phosphoramidate bond in the crosslinked products was cleaved by adding trifluoroacetic acid (TFA) to a final concentration of 50% and incubating at 16° C. for 3 hours. The reaction products were separated by SDS-PAGE and visualized by coomassie staining, silver staining, in-gel fluorescence imaging, or western blotting with a CK2 antibody (Millipore) after transfer onto a PVDF membrane (Immobilon-PSQ, Millipore).

ATP-ArN₃ was synthesized as described in Supporting Information, with characterization by ¹H, ¹³C and ³¹P NMR, UV, and HRMS. The CK2 and Abl substrate peptides were purchased from New England Biolabs (NEB), while the PKA substrate peptide and fluorophore labeled peptides were synthesized by Fmoc-based solid phase peptide synthesis, as described previously. PKA, CK2, and Abl were purchased from NEB. For kinase-catalyzed labeling, each reaction mixture contained either ATP or ATP-ArN₃ (1.3 mM) along with substrate peptide (33.3 μM). Into each reaction mixture, PKA (26.6 units/μL), CK2 (9 units/μL), or Abl (9 units/μL) was added, along with the manufacturers supplied buffer. The reaction mixtures were incubated for 2 hours at 30° C. before quantitative MALDI-TOF MS analysis (Bruker Daltonics), as previously described. For the photocrosslinking experiments, CK2 (50 units/μL, 0.7 μM) was incubated with either Rho-14 (200 μM,) or full length α-casein (100 μM, Sigma), while Abl (20 units/μL, 44.4 nM) was incubated with ROX-13 (200 μM). All reactions also contained ATP or ATP-ArN₃ (1.3 mM) and the manufacturers' kinase buffer.

### Example 2

For example, phosphorylation dependent cross linking of CKII kinase with α-Casein substrate was performed using ATP-Iodoacetamide (ATP-IAc) analog. The ATP-IAc analog is an affinity-based analog that forms a covalent bond with reactive amino acid residues, such as cysteine, tyrosine, histidine and lysine, only when in close proximity. Hence affinity based analog is likely to be more specific compared to ATP-ArN₃, by crosslinking only proteins nearby.

To demonstrate the utility of an affinity-based ATP analog, ATP-IAc was applied towards the crosslinking of CKII kinase and α-Casein. The experiment was carried out similarly to the ATP-ArN₃ experiment, however without UV irradiation. In this case, the ATP-Iodo analog used was used at a higher concentration (25 mM final concentration) because of contamination with ATP, ADP and AMP. The experiment was carried out for 2 hours at 31° C. followed by SDS-PAGE analysis and western blot analysis. Western blot analysis with the anti-CKII antibody revealed the presence of a tight protein band at approx 70 kD, which is consistent with the expected crosslinked band of CKII and α-Casein. This was similar to the crosslinked band observed with ATP-ArN3, however the band was more highly resolved. The more highly resolved band with the ATP-IAc analog suggests that the crosslinking is more specific towards attachment to nearby groups. These studies with the ATP-IAc demonstrate the feasibility and utility of affinity-based crosslinking ATP analogs.

Throughout this application, various publications, including United States patents, are referenced by author and year and patents by number. Full citations for the publications are included. The disclosures of these publications and patents in their entireties are hereby incorporated by reference into this application in order to more fully describe the state of the art to which this invention pertains.

The invention has been described in an illustrative manner, and it is to be understood that the terminology, which has been used, is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1
MALDI-TOF MS analysis of peptides 11-13 after
incubation with ATP-ArN3 and PKA, CK2, or
Abl Kinase.

| Peptide substrate | Kinase | Conversion^{[a]} |
|---|---|---|
| (11) Ac-LRRTSIIGT | PKA | 86% |
| (12) RRREEETEEE | CK2 | 51% |
| (13) EAIYAAPFAKKK | Abl | 78% |

^{[a]}Calculated using quantitative MS by comparing to phosphorylation with ATP (100%).

What is claimed is:

1. An analog comprising adenosine 5′-triphosphate analogs modified at the gamma-phosphate with a reactive reagent.

2. The analog according to claim 1, wherein said adenosine 5′-triphosphate analog is selected from the group consisting essentially of ATP-arylazide, ATP-benzophenone, ATP-diazirine, ATP-azide, ATP-alkyne, bi-functional crosslinking ATP analogs, and ATP-iodoacetamide.

3. The analog according to claim 1 for use in characterizing phosphorylated proteins.

4. The analog according to claim 1 for use in understanding protein-protein interactions.

5. A method of forming the analog according to claim 1 comprising the steps of:
activating a 4-amino benzoic acid;
incubating the activated acid to obtain an amine; and
coupling the amine with ATP in the presence of water soluble EDCI.

6. A kit for detecting characterizing a target protein in a sample, the kit comprising:
a gamma-phosphate modified ATP analog for selectively tagging a protein at phosphorylated amino acids with a cross-linking group.

7. A method of using the kit according to claim 6 to characterize the target protein comprising the steps of:
adding a gamma-phosphate modified ATP analog to a protein substrate;
reacting the target proteins with the ATP analog; and
analyzing the resulting cross-linked product.

8. The method according to claim 7, wherein said analyzing step is performed using a method selected from the group consisting essentially of gel electrophoresis, Western blotting, SDS-PAGE, chromatography, and mass spectrometric analysis.

9. The method according to claim 7, further including the step of detecting abnormally phosphorylated proteins.

10. The method according to claim 9, further including correlating the abnormally phosphorylated protein with a specific disease state.

11. The method according to claim 10, further including the step of identifying potential therapeutics for use in preventing the abnormal phosphorylation and the specific disease state.

12. A method of detecting the efficacy of a therapeutic comprising the steps of:
adding a gamma-phosphate modified ATP analog to a protein substrate;
reacting the target proteins with the ATP analog; and
analyzing the resulting cross-linked product, wherein the amount of product present correlates to the efficacy of the therapeutic.

13. A screen identifying therapeutics, the screen comprising the kit according to claim 6.

14. Use of the ATP-analog of claim 1 for identification of phosphorylation-associated biomolecules.

15. The use according to claim 14, wherein said identification comprises the steps of:
adding a gamma-phosphate modified ATP analog to a protein substrate;
reacting the target proteins with the ATP analog;
analyzing the resulting cross-linked product; and
identifying phosphorylation-associated biomolecules based on results from said analyzing step.


SEQUENCE LISTING (4 sequences)

SEQ ID NO: 1 - protein, 11 aa, Artificial sequence
  synthetic
RRREEETEEEE

SEQ ID NO: 2 - protein, 12 aa, Artificial sequence
  synthetic
EAIYAAPFAKKK

SEQ ID NO: 3 - protein, 9 aa, Artificial sequence
  synthetic
LRRTSIIGT

SEQ ID NO: 4 - protein, 10 aa, Artificial sequence
  synthetic
RRREEETEEE